(19) United States
(12) Patent Application Publication
(10) Pub. No.: US 2022/0105082 A1
(43) Pub. Date: Apr. 7, 2022

(54) NEW FORMULATIONS CONTAINING LEUKOTRIENE RECEPTOR ANTAGONISTS

(71) Applicant: JIANGYIN MUCOCARE PHARMACEUTICAL CO., LTD., Jiangyin, Jiangsu (CN)

(72) Inventor: Bengt Ingemar SAMUELSSON, Solna (SE)

(21) Appl. No.: 17/421,867

(22) PCT Filed: Jan. 10, 2020
(86) PCT No.: PCT/CN2020/071337

§ 371 Date: Jul. 9, 2021

(30) Foreign Application Priority Data
Jan. 10, 2019 (CN) PCT/CN2019/071081

(51) Int. Cl. A61K 31/47; A61K 9/00; A61P 29/00; A61K 9/06; A61P 1/10; A61P 11/00; A61K 45/06

(52) U.S. Cl. CPC A61K 31/47; A61K 9/0014; A61P 29/00; A61K 45/06; A61P 1/10; A61P 11/00; A61K 9/06

(57) ABSTRACT

There is provided pharmaceutical formulations that may be used topically comprising a leukotriene receptor antagonist, a salt or a solvate thereof. Particular leukotriene receptor antagonists that may be mentioned include montelukast styrene. The formulations find particular utility in direct topical administration for the treatment of inflammation, of inflammatory disorders and/or of condition characterized by inflammation, including wounds, burns, psoriasis, haemorrhoids, acne and atopic dermatitis.

### FIELD OF THE INVENTION

This invention relates to novel pharmaceutical combinations and to novel pharmaceutical uses and compositions.

Background and Prior Art

Inflammation is typically characterized as a localised tissue response to e.g. invasion of microorganisms, certain antigens, damaged cells or physical and/or chemical factors. The inflammatory response is normally a protective mechanism which serves to destroy, dilute or sequester both the injurious agent and the injured tissue, as well as to initiate tissue healing.

Inflammation may result from physical trauma, infection, some chronic diseases (e.g. psoriasis and autoimmune diseases, such as rheumatoid arthritis) and/or chemical and/or physiological reactions to external stimuli (e.g. as part of an allergic response). A complex series of events may be involved, in which inflammatory mediators increase blood flow and dilation of local blood vessels, resulting in redness and heat, the exudation of fluids, often resulting in localised swelling, leukocytic migration into the inflamed area, and pain.

Many conditions/disorders are characterized by, and/or are caused by, abnormal, tissue-damaging inflammation. Such conditions are typically characterized by activation of immune defence mechanisms, resulting in an effect that is more harmful than beneficial to the host, and are generally associated with varying degrees of tissue redness or hyperemia, swelling, hyperthermia, pain, itching, cell death, tissue destruction, cell proliferation and/or loss of function. Examples include inflammatory bowel diseases, rheumatoid arthritis, multiple sclerosis, psoriasis, glomerulonephritis and transplant rejection.

Typically, a complex series of events results in inflammatory changes such as increased blood flow through dilation of local blood vessels, resulting in redness and heat, the extravasation of leukocytes and plasma, often resulting in localised swelling, activation of sensory nerves (resulting in pain in some tissues) and loss of function. These inflammatory changes are triggered by a cascade of cellular and biochemical events involving cells like neutrophils, monocytes, macrophages and lymphocytes together with inflammatory mediators such as vasoactive amines, cytokines, complement factors and reactive oxygen species.

Amongst other things, inflammation plays a key role in the wound healing process. Wounds and burns can therefore be classified as conditions with which inflammation is associated. Traditional thinking in the art is that anti-inflammatory drugs should not be applied directly to open wounds, as this would be detrimental to the progress of wound healing.

Mussel adhesive protein (MAP), also known as Mytilus edulis foot protein (mefp), is a protein that is secreted by marine shellfish species, such as Mytilus edulis, Mytilus coruscus and Perna viridis. The adhesive protein is secreted by mussels from the byssus gland where it is produced and stored. When secreted on a surface of a solid, such as a rock, but also other solid objects, such as metals, wood, glass, etc., a water-proof bond is formed which fixes the mussel to the solid object. Mussels are typically attached, in groups, to coastal reefs or to the bottoms of ships. The bond is incredibly strong, having the ability to resist wave impacts in coastal waters.

Studies on Mytilus edulis, Mytilus galloprovincialis, Mytilus californias and Perna viridis have thus far identified eleven separate adhesive protein subtypes derived from mussels: mfp-1 (sometimes referred to as “mefp-1”, hereinafter used interchangeably), mfp-2/mefp-2, mfp-3/mefp-3, mfp-4/mefp-4, mfp-5/mefp-5, mfp-6/mefp-6; the collagens pre-COL-P, pre-COL-D and pre-COL-NG; and the mussel feet matrix proteins PTMP (proximal thread matrix protein) and DTMP (distal proximal thread matrix protein). See, for example, Zhu et al, Advances in Marine Science, 32, 560 (2014) and Gao et al, Journal of Anhui Agr. Sci., 39, 19860 (2011)).

All mussel adhesive proteins, including sub-types thereof, have two structural characteristics, in that they comprise: (1) lysine, such that the protein carries a high positive charge loading (due to the NH₂ termini); (2) 3,4-dihydroxyphenylalanine (DOPA, dopamine), the catechol part of which is responsible for the formation of strong covalent bonds and consequently the ability of mussel adhesive proteins to bind to solid surfaces.

Products based on mussel adhesive protein products are presently used in a limited number of fields (including micro-cellular bonding, as tissue bonding agents and the treatment of wounds and burns). Commercial products are either directly used as a solution of mussel adhesive protein or are stored as a freeze-dried powder for dissolution prior to use.

Leukotriene receptor antagonists (LRAs), often referred to collectively as “leukasts”, including montelukast, are orally-active non-steroidal immunomodulating compounds that are administered perorally to the gastrointestinal tract for the maintenance treatment and prevention of symptoms of seasonal allergies (see e.g. Hon et al, Drug Design, Development and Therapy, 8, 839 (2014)). These compounds act by blocking the action of, primarily, leukotriene D4 (as well as leukotrienes C4 and E4) on the cysteinal leukotriene receptor CysLT1 in the airways.

Although the potential use in the treatment of various other inflammatory disorders has been described in the literature, to the applicant's knowledge, leukotriene receptor antagonists have never been administered topically in the clinical setting, for example to the skin, to treat e.g. inflammation.

Furthermore, to the applicant's knowledge, the use of combination products comprising, specifically, a mussel adhesive protein and a LRA or a derivative thereof to treat, for example, inflammation, has not been disclosed publicly in the prior art.

### DISCLOSURE OF THE INVENTION

According to three first aspects of the invention, there is provided a (pharmaceutical, veterinary or cosmetic) formulation comprising a LRA or a pharmaceutically-, veterinarily- or cosmetically-acceptable salt or solvate thereof, which formulation is suitable for, adapted for, and/or packaged and presented for:
(a) topical administration;
(b) the treatment of inflammation, an inflammatory disorder and/or a condition characterized by inflammation; and/or
(c) use in the treatment of inflammation, an inflammatory disorder and/or a condition characterized by inflammation by way of direct topical administration of that formulation.

LRAs that may be mentioned include cinalukast and pobilukast, preferably pranlukast and zafirlukast, and more preferably a derivative of montelukast, which derivative is of formula I:

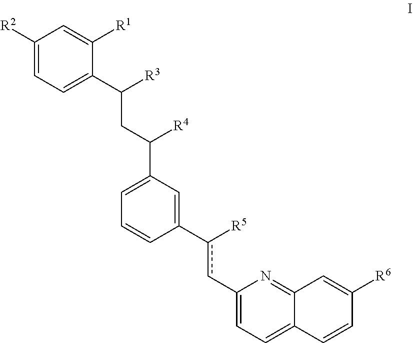

wherein:
R¹ is selected from the group consisting of —C(CH₃)₂OR⁷, —C(═O)CH₃, —C(CH₃)═CH₂, —C(CH₃)₂H, —C(CH₃)(OH)CO₂H, —C(CH₃)(OH)CH₂OH and —C(═O)NH₂;
R² and R³ are independently H or —OH;
R⁴ is H, —OH, —OS(O)₂CH₃, a structural fragment of formula II:

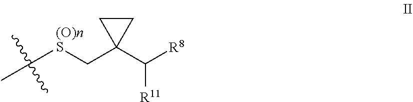

or a structural fragment of formula III:

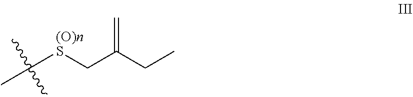

wherein the squiggly lines in the fragments of formula II and III represent the points of attachment of the respective fragments to the compound of formula I, and n is 0, 1 or 2;
the dotted line in the compound of formula I represents an optional double bond and, when a double bond is present, R⁵ represents H and, when a double bond is not present, R⁵ represents H or a structural fragment of formula II as defined above; R⁶ is H or Cl;
R⁸ is selected from the group consisting of —C(O)R⁹, —CN and a structural fragment of formula IV:

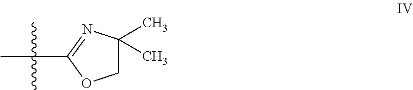 [SMILES: *C1=NC(C)(C)CO1]

wherein the squiggly line in the fragment of formula IV represent the point of attachment to the structural fragment of formula II,
R⁹ is —NH₂ or —OR¹⁰;

### R¹¹ is H or OH; and

R⁷ and R¹⁰ are independently H, —CH₃ or a glucuronide residue,
or a regioisomer, a geomeric isomer, or a stereoisomer, thereof.

As referred to above, when compounds of formula I contain a double bond they may exist as E (entgegen) and Z (zusammen) geometric isomers about the double bond. All such isomers and mixtures thereof are included within the scope of the invention. For the avoidance of doubt, in compounds of formula I, the quinoline ring may be located either cis (as the Z geometric isomer) or trans (as the E geometric isomer) across the double bond to the central 1,3-disubstituted phenyl ring.

Preferably the quinoline ring is located trans across the double bond to the central 1,3-disubstituted phenyl ring i.e. the E geometric isomer.

LRAs and, in particular compounds of formula I, contain chiral carbon atoms. In this respect, all stereoisomers and mixtures (including racemic mixtures) thereof are included within the scope of the invention.

Preferred compounds of formula I include those in which, when R³ represent —OH it is in the following configuration:

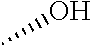

and, more preferably, when R⁴ is a structural fragment of formula II, it is in the following configuration:

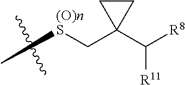

wherein n, R⁸ and R¹¹ are as hereinbefore defined.

Although in some aspects of the invention, the LRA can be montelukast, LRAs that may be mentioned include those in which the LRA is not montelukast. Compounds of formula I that may be mentioned include those as hereinbefore defined, provided that when R¹ is —C(CH₃)₂OR⁷, R⁶ is Cl, R², R³ and R⁷ are all H, the dotted line represents a double bond and the quinoline ring is located trans (as the E geometric isomer) across the double bond to the central 1,3-disubstituted phenyl ring, and R⁴ is a structural fragment of formula II in which n is 0, R¹¹ is H, R⁸ is —C(O)R⁹ and R⁹ is —OR¹⁰, then R¹⁰ is not H.

Preferred compounds of formula I include those in which:

R¹ is selected from the group consisting of —C(CH₃)₂OR⁷, —C(═O)CH₃, —C(CH₃)═CH₂ and —C(CH₃)₂H;

### R² is H;

### R³ is H;

R⁴ is a structural fragment of formula II;
n is 1 or, preferably, 0;
the dotted line represents a double bond;

### R⁶ is Cl;

### R⁷ is H;

R⁸ is —C(O)R⁹;
R⁹ is —OR¹⁰;
R¹⁰ and R¹¹ are each independently H.

Compounds of formula I that may be mentioned include those in which R¹ is selected from the group consisting of —C(CH₃)₂OH, —C(═O)CH₃, —C(CH₃)═CH₂ and —C(CH₃)₂H.

Preferred compounds of formula I include those in which R¹ is —C(CH₃)═CH₂ or —C(CH₃)H, and/or n is 0.

Particularly preferred compounds of formula I include montelukast styrene and hydrogenated montelukast styrene.

For the avoidance of doubt, montelukast styrene has the following chemical structure:

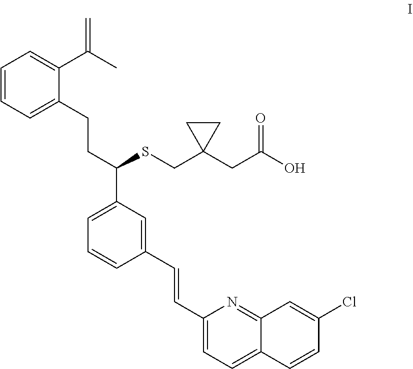 [SMILES: C=C(C)c1ccccc1CC[C@@H](SCC1(CC(=O)O)CC1)c1cccc(/C=C/c2ccc3ccc(Cl)cc3n2)c1]

(see, for example, Saravanan et al, Journal of Pharmaceutical and Biomedical Analysis, 48, 708 (2008)), and hydrogenated montelukast styrene has the following chemical structure:

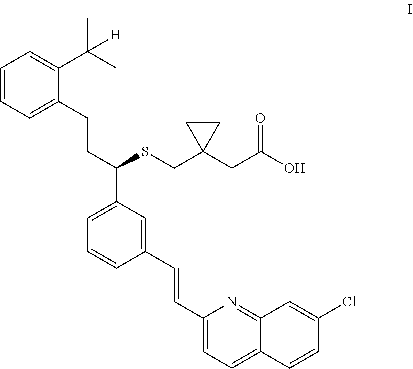 [SMILES: CC(C)c1ccccc1CC[C@@H](SCC1(CC(=O)O)CC1)c1cccc(/C=C/c2ccc3ccc(Cl)cc3n2)c1]

(see, for example, Gandhi et al, Anal. Methods, 8, 1667 (2016)).

LRAs, including compounds of formula I, may be in the form of salts. Salts that may be mentioned include pharmaceutically-acceptable and/or cosmetically-acceptable salts, such as pharmaceutically- and/or cosmetically-acceptable acid addition salts and base addition salts. Such salts may be formed by conventional means, for example by reaction of the relevant compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of active ingredient in the form of a salt with another counter-ion, for example using a suitable ion exchange resin.

Preferred salts include, for example, acetate, hydrochloride, bisulfate, maleate, mesylate, tosylate, alkaline earth metal salts, such as calcium and magnesium, or alkali metal salts, such as sodium and potassium salts. Most preferably, compounds of the invention may be in the form of acetate salts.

The specific LRAs cinalukast, pobilukast, pranlukast and zafirlukast are known LRA active ingredients.

Derivatives of montelukast that are compounds of formula I are known in the literature (see, for example, the literature references described above, as well as Drug Metabolism and Disposition, 43, 1905 (2015) and Toxicology Letters, http://dx.doi.org/10.1016//j.tox.let.2015.07.03), are available commercially, and/or may be prepared by way of conventional techniques from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006. See also syntheses described in inter alia Saravanan et al, Journal of Pharmaceutical and Biomedical Analysis, 48, 708 (2008), Gandhi et al, Analytical Methods, 8, 1667 (2016) and Cardoso et al, Drug Metab. Dispos., 43, 1905 (2015).

The skilled person will understand that the substituents as defined herein, and substituents thereon, may be modified one or more times, after or during processes for the preparation of compounds of formula I by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. Precursor groups may be changed to a different such group, or to the groups defined in a compound of formula I, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds of formula I may be isolated from their reaction mixtures and, if necessary, purified using conventional techniques as known to those skilled in the art. Thus, processes for the preparation of compounds of formula I may include, as a final step, isolation and optionally purification of the relevant compound.

It will be appreciated by those skilled in the art that, in processes for the preparation of compounds of formula I, the functional groups of intermediate compounds may need to be protected by protecting groups. The protection and deprotection of functional groups may take place before or after a reaction.

Protecting groups may be applied and removed in accordance with techniques that are well-known to those skilled in the art and as described hereinafter. For example, protected compounds/intermediates may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 5th edition, T. W. Greene & P. G. M. Wutz, Wiley-Interscience (2014), the contents of which are incorporated herein by reference.

Certain compounds of formula I are useful because they possess pharmacological activity. Thus, those compounds are useful in human and animal medicine. They are therefore indicated as pharmaceuticals (and/or in veterinary science), although they may also be used as cosmetics and/or as part of a medical device.

Thus, in a further aspect of the invention, there is provided a pharmaceutical, veterinary or cosmetic formulation comprising a compound of formula I as hereinbefore defined, or a pharmaceutically acceptable salt or solvate thereof, which formulation is suitable for, adapted for, and/or packaged and presented for administration to humans or animals. In this respect, in such a formulation, a compound of formula I as hereinbefore defined, or a pharmaceutically acceptable salt or solvate thereof, is present in an amount that is greater than about 10%, such as greater than about 20%, including greater than about 40%, by weight of the total amount of all active pharmaceutical ingredients that are present in the formulation.

Preferred pharmaceutical formulations comprising compounds of formula I include those comprising montelukast styrene.

There is further provided a compound of formula I as hereinbefore defined (e.g. montelukast styrene), or a pharmaceutically-acceptable salt or solvate thereof, for use as a pharmaceutical medicine, a veterinary medicine or a cosmetic.

Although LRAs described herein may possess pharmacological activity as such, certain pharmaceutically-acceptable (e.g. “protected”) derivatives thereof may exist or may be prepared which may not possess such activity, but which may be administered and thereafter be metabolised or chemically transformed to form such LRAs. Such compounds (which may possess some pharmacological activity, provided that such activity is appreciably lower than that of the active compounds to which they are metabolised/transformed) may therefore be described as “prodrugs” of the LRAs described herein.

As used herein, references to prodrugs will include compounds that form a relevant LRA compound in an experimentally-detectable amount, within a predetermined time, following administration. All prodrugs of the LRA compounds described herein are included within the scope of the invention.

The LRAs described herein are particularly useful in the treatment of inflammation.

The “treatment of inflammation” includes the treatment of inflammation in any organ of the body (including soft tissue, joints, nerves, the vascular system, internal organs, especially mucosal surfaces, and particularly the skin), irrespective of the cause, and also includes all such inflammatory disorders or conditions, and/or disorders or conditions characterized by inflammation (e.g. as a symptom).

Inflammatory disorders and/or conditions may be (and are typically) characterized by activation of immune defence mechanisms, resulting in an effect that is more harmful than beneficial to the host. Such conditions are generally associated with varying degrees of tissue redness or hyperemia, swelling, edema, hyperthermia, pain (including aching), exudation of body fluids, itching (pruritis), cell death and tissue destruction, cell proliferation, and/or loss of function.

According to a further aspect of the invention there is provided a method of treatment of inflammation, of an inflammatory disorder, and/or of a disorder/condition characterized by inflammation (for example as a symptom), which method comprises the administration of a formulation comprising a LRA or a salt or solvate thereof as disclosed herein to a patient in need of such treatment.

Inflammatory conditions that may be mentioned include arteritis, diabetes mellitus, metabolic syndrome, rosacea, asthma and allergy, ankylosing spondylitis, chronic obstructive pulmonary disease, gouty arthritis, inflammatory bowel disease (such as Crohn's disease and ulcerative colitis), multiple sclerosis, osteoarthritis, pancreatitis, prostatitis, psoriatic arthritis, rheumatoid arthritis, tendinitis, bursitis, Sjogren's syndrome, systemic lupus erythematosus, uveitis, urticaria, vasculitis, mastocytosis, diabetic vascular complications, migraine, atherosclerosis and associated cardiovascular disorders. A disease state characterized by inflammation that may be mentioned is chronic obstructive pulmonary disease (COPD). A further disease state characterized by inflammation that may be mentioned is colitis, including inflammatory bowel diseases including Crohn's disease and, especially, ulcerative colitis. Other disease states characterized by inflammation that may be mentioned are gastrohelcosis (e.g. gastritis, gastric ulcer, gastric cancer and other stomach mucosa diseases), constipation, gastritis, inflammation associated with cancers and infections (e.g. viral infections, such as the common cold or influenza) that affect the gastrointestinal tract, and gastroesophageal reflux disease (GERD).

Inflammatory conditions that may be more especially mentioned include inflammations of the skin or mucosa (including the oral, nasal, ocular, vaginal, cervical and/or anorectal mucosa, more particularly the oral or nasal mucosae), such as inflammation resulting from infections (such as viral and/or bacterial infections), or allergic/atopic conditions (such as rhinitis (e.g. allergic rhinitis), pharyngitis, periodontitis, gingivitis, xerophthalmia, conjunctivitis (e.g. allergic conjunctivitis), dermatitis, urticaria (hives) and food allergy); and other inflammatory conditions, such as herpes, drug eruptions, polymorphous light eruptions, sunburn, early manifestations of skin cancers (erythema-like skin lesions), pathological hair loss (including following skin grafting), chemo rash, psoriasis, erythema multiforme, folliculitis, eczema and external otitis. A disease state that may be mentioned is polymorphous light eruptions.

More particularly, formulations (e.g. topical formulations) comprising LRA compounds may be used to treat certain conditions characterized by inflammation, and/or with which inflammation is associated. Such conditions may include wounds (including abrasions (scratches), incisions (including operative incisions), lacerations, punctures, avulsions, bruising and scarring), and burns (including inflammation resulting from surgery following burns, such as skin grafting) and other conditions, such as hemorrhoids. Wounds may be acute or chronic, and/or may result from one or more inflammatory disorders as defined herein.

Wounds of the skin or mucosa may arise from internal or external physical injury to the membrane surface, or may be caused by (i.e. be a symptom of) an underlying physiological disorder.

Physical (e.g. “open”) wounds may be caused by sharp objects (cuts, incisions, punctures) or blunt objects/mechanical forces (lacerations, abrasions, avulsions), physical blows (bruises), heat or chemicals (burns and blisters), UV light (sunburn), cold (chilblains or frostbite). Wounds may be superficial (damage only to the epidermis and/or dermis) or may be full thickness wounds (damage below the epidermis and/or dermis). In serious cases, subcutaneous and/or submucosal tissues, such as muscles, bones, joints, and even internal organs, may be damaged.

Formulations (e.g. topical formulations) comprising LRA compounds may be used to relieve the pain (including aching) associated with inflammation and/or wounding. In particular, the formulations comprising LRAs disclosed herein may be used to relieve procedural pain and/or non-procedural pain. The skilled person will understand that the term “procedural pain” (i.e. operation pain) refers to acute pain that is associated with medical investigations and treatments conducted for the purpose of healthcare. The term “non-procedural” refers to general pain that is associated with inflammation and/or wounding (e.g. pain associated with dental ulcers, burns and/or scars), and is not a consequence of a particular medical intervention.

The formulations comprising LRA compounds disclosed herein may be used to treat not only the inflammation, pain (including aching) and/or pruritis (itching) associated with the wound itself and the healing process, but also they may be used to prevent the exudation of body fluids from wounds, the risk of infection, and also the prevention of physiological reactions that result from inflammation and/or wound healing processes, such as scarring and melanin pigmentation.

Scarring is a consequence of inflammation and/or wound healing and is a general term for the formation of fibrotic tissue that is a consequence of such inflammation/healing.

Formulations (e.g. topical formulations) comprising LRA compounds disclosed herein may also be useful in the suppression of the production of melanin pigmentation which may or may not result from inflammation and/or wound healing. Such formulations may also be useful in the suppression of disorders associated with melanin pigmentation, such as chloasma, freckles, melanosis, malar rash and other chromatosis, skin cancers with melanoma, and chromatosis that is caused by exposure to the sun or skin diseases like acne.

Wounds may also arise as a consequence of (e.g. inflammatory) diseases or disorders. Such wounds may include blistering and/or ulcers of the skin and mucosa. These are common conditions that are often long-lasting and difficult to treat. Skin tissues can often be damaged, removed, liquefied, infected and/or necrotic. Ulcers can lead to secondary consequences to health particularly if they become infected, are hard to heal and are costly to treat. They can also cause significant psychological stress and economic loss to patients, affecting both general well-being and quality of life.

According to a further aspect of the invention, there is provided the use of a pharmaceutical formulation suitable for, adapted for, and/or packaged and presented for, topical administration, comprising a LRA, or a pharmaceutically acceptable salt or solvate thereof, in admixture with a pharmaceutically-acceptable topical adjuvant, diluent or carrier, for the manufacture of a medicament for the healing or the recovery of a wound of the skin or of the mucosa, by direct topical application of said formulation to said wound.

In the alternative, inflammatory skin conditions or diseases in which formulations (e.g. topical formulations) comprising LRA compounds disclosed herein find particular utility include psoriasis, acne, eczema and dermatitis, especially allergic/atopic dermatitis, as well as in the treatment of, for example, mucosal inflammation as characterized by rhinitis, especially allergic rhinitis, hemorrhoids and chronic obstructive pulmonary disease, and also ulcerative colitis, for example.

Psoriasis is a chronic, inflammatory skin disease with a tendency to recur (some patients never heal during their entire life). Clinical manifestations of psoriasis mainly include erythema and scales. It can occur over the whole body, but is more commonly observed on the scalp and limbs.

Acne is a follicular (pilosebaceous unit) chronic, inflammatory skin disease, the occurrence of which is closely related to main factors like hypersteatosis, blocked pilosebaceous ducts (including closed and open comedones), bacterial infection and inflammatory reactions, that tends to occur during youth, characterized by multiform skin lesions on the face. The term acne thus includes regular acne and acne rosacea (i.e. copper nose).

Eczema is a skin inflammatory reaction with strong itching caused by a variety of internal and external factors. It has three phases, acute, sub-acute, and chronic. In the acute phase, there is a tendency for the production of exudates, while the chronic phase includes infiltration and hypertrophy. Skin lesions are often itchy and recur easily.

Dermatitis is a common skin disease characterized by coarseness, redness, itching, eczema, and dryness. Small lumps, refractory ulcers, and pigmented spots caused by dermatitis may, if not treated promptly, develop to basal cell carcinoma, squamous cell carcinoma, and malignant melanoma. Dermatitis may be caused by various internal and external infectious or non-infectious factors, including substances (contact dermatitis) or allergy (allergic/atopic dermatitis). Also included is seborrheic dermatitis (seborrheic eczema) and all forms of steroid-dependent dermatitis (including light-sensitive seborrheid, perioral dermatitis, rosacea-like dermatitis, steroid-rosacea, steroid-induced rosacea, iatrosacea, steroid dermatitis resembling rosacea, topical corticosteroid-induced rosacea-like dermatitis and, more particularly, facial corticosteroid addictive dermatitis (FCAD) or facial corticosteroid-dependent dermatitis (FCDD), as characterized by flushing, erythema, telangiectasia, atrophy, papules and/or pustules in the facial area after long-term treatment with (including uncontrolled use, abuse or misuse of) topical corticosteroids; see, for example, Xiao et al, J. Dermatol., 42, 697 (2015) and Lu et al, Clin. Exp. Dermatol., 35, 618 (2009)).

Rhinitis is irritation and inflammation of the mucous membrane inside the nose. Common symptoms of rhinitis include a stuffy nose, runny nose, sneezing and post-nasal drip. The most common kind of rhinitis is allergic rhinitis, caused by an allergen, such as pollen, dust, mould, or flakes of skin from certain animals. The formulations comprising LRA compounds disclosed herein may give rise to relief of eye itchiness, even when administered nasally (i.e. to the nasal mucosa).

Hemorrhoids are swellings caused by inflammation of the hemorrhoidal blood vessels found inside or around the rectum and the anus. Symptoms include bleeding (i.e. wounding) after the passage of a stool, prolapse of the hemorrhoid, mucus discharge and itchiness, soreness, redness and swelling in the area of the anus. Hemorrhoids are believed to be a consequence of an increase of pressure in the abdomen, for example, as a result of constipation or diarrhea.

Chronic obstructive pulmonary disease (COPD) is the name for a group of lung conditions that cause breathing difficulties, including emphysema (damage to the alveoli) and chronic bronchitis (long-term inflammation of the airways). COPD occurs when the lungs become inflamed, damaged and narrowed. The damage to the lungs is usually irreversible and results in an impairment of the flow of air into and out of the lungs. Symptoms of COPD include breathlessness, productive cough, frequent chest infections and persistent wheezing. The most common cause of the disease is smoking, although other risk factors include high levels of air pollution and occupational exposure to dust, chemicals and fumes.

Formulations (e.g. topical formulations) comprising LRA compounds disclosed herein may have positive effects in mitigating erythema, redness and swelling, edema, blisters, and bullous pemphigoid caused by various conditions including those mentioned generally and specifically herein, and may inhibit exudation of subcutaneous tissue fluid, and suppressing itching and pain caused by such inflammatory conditions.

Other inflammatory conditions that may be mentioned include:

(a) Mucosal inflammation, such as oral mucositis, aphthous ulcers, otitis media, laryngitis, tracheitis, esophagitis, gastritis, enteritis and enterocolitis (including bacillary dysentery, chronic amoebic dysentery, schistosomiasis, nonspecific ulcerative colitis and regional enteritis), cervicitis and endocervicitis, endometritis, inflammation caused by inhalation injury and the like, as well as mucosal inflammation associated with cancers, and infections (e.g. viral infections, such as the common cold or influenza), that affect mucosal surfaces, such as those in the oral cavity, the nasopharynx, the ear, the throat, the trachea, the gastrointestinal tract, the cervix, etc.
(b) Orthopedic inflammation associated with, for example bone fractures, pyogenic infection of bones and joints, inflammation caused by rheumatic bone diseases, as well as pyogenic osteomyelitis (acute, chronic, localized, sclerotic, post-traumatic), pyogenic arthritis; bone tumors (osteoma, osteoid osteoma, chondroma), bone cysts, osteoclastoma, primary bone sarcoma (osteosarcoma, chondrosarcoma, osteofibrosarcoma, Ewing's sarcoma, non-Hodgkin's lymphoma, myeloma, chordoma), metastatic bone tumors, tumor-like lesions of bone (bone cyst, aneurysmal bone cyst, eosinophilic granuloma, fibrous dysplasia); and rheumatic arthritis.
(c) Nerve inflammation, such as peripheral polyneuritis, facial neuritis, peripheral neuritis, subcutaneous neuritis, ulnar neuritis, intercostal neuritis, etc.
(d) Subcutaneous and submucosal soft tissue inflammation, such as myositis, ligamentitis, tendonitis, panniculitis capsulitis, lymphadenitis, bubonadentitis, tonsillitis, synovitis, fasciitis, and soft tissue inflammation caused by injuries, contusion or laceration of muscles, ligaments, fascia, tendons, membrana synovialis, fat, articular capsules, and lymphoid tissue.
(e) Vascular inflammation, such as allergic leukocytoclastic vasculitis, allergic cutaneous vasculitis, polyarteritis nodosa, thrombotic vasculitis, granulomatous vasculitis, lymphocytic vasculitis, vasculitis with abnormalities in blood composition, and rheumatic vasculitis, as well as vascular inflammation associated with vascular cancers caused by allergic leukocytoclastic vasculitis, polyarteritis nodosa, thrombotic vasculitis, granulomatous vasculitis, lymphocytic vasculitis, vasculitis with abnormalities in blood composition, and rheumatic vasculitis.
(f) Inflammation of the internal organs, such as the heart, stomach, intestine, lung, liver, spleen, kidney, pancreas, bladder, ovary, and prostate, including but not limited to pericarditis, myocarditis, endocarditis, pneumonia, hepatitis, splenitis, nephritis pancreatitis, cystitis, oophoritis, prostatitis and treatment of gastric ulcer.
(g) Inflammation of the eye and surrounding area, such as conjunctivitis, keratitis (e.g. acute epithelial keratitis, nummular keratitis, interstitial keratitis, disciform keratitis, neurotrophic keratitis, mucous plaque keratitis, herpes simplex keratitis, herpes zoster keratitis, bacterial keratitis, fungal keratitis acanthamoebic keratitis, onchocercal keratitis, superficial punctate keratitis, ulcerative keratitis, exposure keratitis photokeratitis and contact lens acute red eye), optic neuritis, etc.
(h) Inflammation of the gums and the oral cavity, such as periodontitis, gingivitis, dental ulcers, etc.
(i) Inflammation associated with rheumatism, such as rheumatic vasculitis, rheumatoid arthritis, rheumatic bone diseases, ankylosing spondylitis, bursitis, Crohn's disease, gout, infectious arthritis, juvenile idiopathic arthritis, osteoarthritis, osteoporosis, polymyalgia rheumatica, polymyositis, psoriatic arthritis, scleroderma, Sjögren's syndrome, spondyloarthropathies, systemic lupus erythematosus, tendinitis, etc.

Compounds of the invention may also be used in the treatment of certain specific diseases of the digestive system, such as gastroesophageal reflux disease (GERD), which may be characterised by an acidic taste in the mouth, regurgitation, heartburn, pain with swallowing and/or sore throat, increased salivation (water brash), nausea, chest pain, and coughing. GERD may cause injury of the esophagus, including reflux esophagitis (i.e. inflammation of the esophageal epithelium which may cause ulceration at or around the junction of the stomach and esophagus), esophageal strictures (i.e. the persistent narrowing of the esophagus caused by reflux-induced inflammation), Barrett's esophagus (i.e. intestinal metaplasia (i.e. changes of epithelial cells from squamous to intestinal columnar epithelium of the distal esophagus) and/or esophageal adenocarcinoma (a form of cancer).

The formulations comprising LRA compounds disclosed herein may also be used in the treatment of certain specific diseases of the respiratory system, such as pulmonary cystic fibrosis, usual interstitial pneumonia, allergic pneumonia, asbestosis, emphysema, pulmonary heart disease, pulmonary embolism, etc. A specific disease state that may be mentioned in idiopathic pulmonary fibrosis (IPF).

IPF is a diffuse and fatal pulmonary interstitial disease with pathological features including alveolar epithelial damage, massive proliferation of lung fibroblasts, excessive deposition of extracellular matrix, ultimately leading to irreversible lung tissue damage. In the latter stages of the disease, subjects with IPF experience respiratory failure and death. It has been found that formulations comprising LRA compounds disclosed herein may find utility in the treatment of IPF and/or alleviation of the symptoms associated with the disease.

The formulations comprising LRA compounds disclosed herein may also be particularly useful in the treatment of the following lung and/or fibrotic conditions (whether otherwise mentioned herein or not): lung fibrosis, renal fibrosis, liver fibrosis, silicosis, acute bronchitis, chronic bronchitis, tracheobronchitis, bronchial asthma, status asthmatics, bronchiectasis, upper respiratory tract infections, including the common cold and influenza), allergic airway inflammation, bacterial pneumonia, viral pneumonia, mycoplasma pneumonia, reckettsia, radiaton pneumonia, pneumococcal (including staphylococcal, streptococcal and gram-negative bacillus) pneumonia, pulmonary candidiasis (including aspergillosis, mucormycosis, histoplasmosis, actinomycosis and nocardiosis), pulmonary mycosis, cryptococcosis, lung abscesses, anaphylactic pneumonia (Leoffer's syndrome), extrinsic allergic alveolitis, pulmonary eosinophia (eosinophilosis), obstructive pulmonary emphysema, pulmonary edema, pulmonary tuberculosis, respiratory alkalosis (acidosis), acute lung injury, interstitial lung disease, empyema, lung fibroma and cor pulmonale.

According to a further aspect of the invention, there is provided the use of a pharmaceutical formulation suitable for, adapted for, and/or packaged and presented for, topical administration, comprising a LRA, or a pharmaceutically acceptable salt or solvate thereof, in admixture with a pharmaceutically-acceptable topical adjuvant, diluent or carrier, for the manufacture of a medicament for the treatment of IPF by topical administration to the lung (e.g. by inhalation).

Particular mucosal disorders and diseases in which formulations comprising LRA compounds disclosed herein find utility include anorectal diseases, such as diarrhea, hemorrhoids, abscesses, fistula, fissures, anal itching, anal sinusitis, warts and rectal prolapse; inflammatory bowel disease, including Crohn's disease and, particularly, ulcerative colitis; gynaecological diseases, such as cervicitis, vaginitis, pelvic pain and disorders; and dental diseases, such as paradentitis, for example.

LRA compounds defined herein may further possess an antioxidation effect, by increasing SOD (superoxide dismutase) production and reducing lipid oxidation. Such compounds and formulations including them may therefore be considered to have antioxidant properties.

LRA compounds defined herein may also possess antipyretic properties that allow for the treatment of a fever and/or alleviate the symptoms thereof; for example, by reducing a subject's body temperature, which results in a reduction of fever. Such LRA compounds and formulations including them may therefore be considered to be antipyretics.

For the avoidance of doubt, in the context of the present invention, the terms “treatment”, “therapy” and “therapy method” include the therapeutic, or palliative, treatment of patients in need of, as well as the prophylactic treatment and/or diagnosis of patients which are susceptible to, inflammation and/or inflammatory disorders.

The formulations comprising LRA compounds and salts thereof as disclosed herein may further possess antiviral properties that may allow for the treatment of a viral infection per se, that is treatment of a viral infection, or a viral disease, by interfering with the replication of the virus within a host, as opposed to the treatment of any symptoms of any viral infection or disease, such as pain and/or inflammation. Such antiviral properties may also allow for the prevention of the onset of such an infection or disease, the protection of cells in a host from (e.g. further) viral infection, prevention or arrest of the spread of viral infection or disease (within a single host, or from one host to a new host), or for the prevention of reactivation of a virus after latency in a host.

According to a further aspect of the invention there is provided a method of treatment of a viral infection, which method comprises the administration of a formulation comprising a LRA or a salt or solvate thereof as disclosed herein to a patient in need of such treatment.

Viral infections that may be mentioned include those caused by viruses in the following families: adenoviridae (e.g. adenovirus), papillomaviridae (e.g. human papillomavirus), polyomaviridae (e.g. BK virus; JC virus), herpesviridae (e.g. herpes simplex, type 1; herpes simplex, type 2; varicella-zoster virus; Epstein-Barr virus; human cytomegalovirus; human herpes virus, type 8), poxviridae (e.g. smallpox), hepadnaviridae (e.g. hepatitis B virus), parvoviridae (e.g. parvovirus B19), astroviridae (e.g. human astrovirus), caliciviridae (e.g. norovirus; Norwalk virus), picornaviridae (e.g. coxsackievirus, hepatitis A virus; poliovirus; rhinovirus), coronoviridae (e.g. severe acute respiratory syndrome virus), flaviviridae (e.g. hepatitis C virus; yellow fever virus; dengue virus; West Nile virus; tick-borne encephalitis virus), retroviridae (e.g. human immunodeficiency virus; HIV), togaviridae (e.g. rubella virus), arenaviridae (e.g. Lassa virus), bunyaviridae (e.g. hantavirus; Crimean-Congo hemorrhagic fever virus; Hantaan virus), filoviridae (e.g. Ebola virus; Marburg virus; Ravn virus), orthomyxoviridae (e.g. influenza viruses, including influenza A virus (e.g. H1N1 and H3N2 viruses), influenza B virus or influenza C virus), paramyxoviridae (e.g. measles virus; mumps virus; parainfluenza virus, respiratory syncytial virus), rhabdoviridae (e.g. rabies virus), hepeviridae (e.g. hepatitis E virus), reoviridae (e.g. rotavirus; orbivirus; coltivirus; Banna virus), as well as viruses not assigned to families, such as hepatitis D virus.

Viruses that may be more specifically mentioned include herpes simplex, type 1 and herpes simplex, type 2 viruses, human papillomavirus, influenza virus and parainfluenza virus.

Formulations comprising LRA compounds and salts thereof as disclosed herein may further possess antibacterial and/or bacteriostatic properties that may allow for the treatment of a bacterial infection per se, that is treatment of a bacterial infection, or a bacterial disease, by interfering with bacterial growth or proliferation in a host, as opposed to the treatment of any symptoms of any bacterial infection or disease, such as pain and/or inflammation. LRA compounds, salts thereof, and formulations containing them, may therefore be considered to be bacteriocides and/or, preferably, bacteriostatic agents.

Such antibacterial properties may also allow for the prevention of the onset of such an infection or disease, the protection of cells in a host from (e.g. further) bacterial infection, prevention or arrest of the spread of bacterial infection or disease (within a single host, or from one host to a new host), or for the prevention of reactivation of a bacterium after latency in a host.

According to a further aspect of the invention there is provided a method of treatment of a bacterial infection, which method comprises the administration of a formulation comprising a LRA or a salt or solvate thereof as disclosed herein to a patient in need of such treatment.

Formulations comprising LRA compounds and salts thereof as disclosed herein may further possess anticancer properties that may allow for the treatment of a cancer per se, that is treatment of a cancer by interfering with the cancer as opposed to the treatment of any symptoms of the cancer, such as pain and/or inflammation. Such anticancer properties may also include the prevention of the onset of such a disease e.g. by treating inflammation and thereby preventing such onset.

According to another aspect of the invention, there is provided a method of treatment of cancer, which method comprises the administration of a formulation comprising a LRA or a salt or solvate thereof as disclosed herein to a patient in need of such treatment.

Particular cancers that may be mentioned include oral cancer, a nasopharynx cancer, a middle ear cancer, a conjunctival cancer, a throat cancer, a tracheal cancer, an esophageal cancer, a gastric cancer, an intestinal cancer, a cervical cancer, an endometrial cancer, skin cancer and the like caused by oral mucositis, rhinitis, otitis media, conjunctivitis, pharyngitis, laryngitis, tracheitis, esophagitis, gastritis, enterocolitis, cervicitis, endometritis, erythema-like skin lesions and the like. A particular skin cancer that may be mentioned is basal cell carcinoma.

“Patients” include reptilian, avian and, preferably, mammalian (particularly human) patients.

In accordance with the invention, formulations comprising LRA compounds and salts thereof as disclosed herein are preferably administered locally or systemically, for example orally, intravenously or intraarterially (including by intravascular and other perivascular devices/dosage forms (e.g. stents)), intramuscularly, cutaneously, subcutaneously, transmucosally (e.g. sublingually or buccally), rectally, intravaginally, intradermally, transdermally, nasally, pulmonarily (e.g. tracheally or bronchially), preferably topically, or by any other parenteral route, in the form of a pharmaceutical preparation comprising the relevant compound(s) in pharmaceutically acceptable dosage form(s).

Administration by inhalation (e.g. nasally) is particularly useful when the condition to be treated is rhinitis or inflammation resulting from viral infections of the airways (common cold, influenza).

Pulmonary administration is particularly useful when the condition to be treated is COPD or IPF. Topical forms of administration may be enhanced by creating a spray comprising active ingredients, e.g. by using a powder aerosol or by way of an aqueous mist using an appropriate atomisation technique or apparatus, such as a nebulizer.

Anorectal administration is particularly useful when the condition to be treated is hemorrhoids or ulcerative colitis, using an appropriate delivery means, such as a solution of foam to be injected or a suppository.

Administration to the lower gastrointestinal tract may also be achieved by parenteral, and particularly by peroral, delivery, by means of standard delayed- or extended-release coating techniques known to those skilled in the art. In particular, distinct parts of the upper or lower intestine may be targeted. For example, colonic administration can also be achieved by way of colon-targeted drug delivery means that are initially administered perorally or parenterally.

Preferred modes of delivery of LRA compounds and salts thereof as disclosed herein include topically to the site of inflammation (e.g. the mucosa, including the oral and/or nasal mucosa, the lung, the anorectal area and/or the colon, or, more preferably, the skin) in an appropriate (for example a pharmaceutically- and topically-acceptable) vehicle suitable for application to the skin and/or the appropriate mucosal surface, and/or a commercially-available formulation, but may also include oral, intravenous, cutaneous or subcutaneous, nasal, intramuscular, intraperitoneal, or pulmonary delivery.

Administration by intradermal injection (e.g. intradermally) is particularly useful for administering the active ingredient, in the form of a solution or suspension (e.g. a dermal filler), into the dermis.

Formulations of the invention comprise relevant LRAs or salts thereof in admixture with a (e.g. pharmaceutically acceptable) adjuvant, diluent or carrier, which may be selected with due regard to the intended route of administration (e.g. topical to the relevant mucosa (including the lung) or, preferably, the skin) and standard pharmaceutical or other (e.g. cosmetic) practice. Such pharmaceutically acceptable carriers may be chemically inert to the active compounds and may have no detrimental side effects or toxicity under the conditions of use. Such pharmaceutically acceptable carriers may also impart an immediate, or a modified, release of the active ingredient.

Suitable pharmaceutical formulations may be commercially available or otherwise prepared according to techniques that are described in the literature, for example, Remington The Science and Practice of Pharmacy, 22nd edition, Pharmaceutical Press (2012) and Martindale—The Complete Drug Reference, 38th Edition, Pharmaceutical Press (2014) and the documents referred to therein, the relevant disclosures in all of which documents are hereby incorporated by reference. Otherwise, the preparation of suitable formulations including LRAs/salts may be achieved non-inventively by the skilled person using routine techniques.

A formulation of the invention may be an aqueous formulation such as an emulsion, a suspension and/or a solution (e.g. an (optionally) buffered aqueous formulation (e.g. solution), such as a physiological saline-containing formulation (e.g. solution), a phosphate-containing formulation (e.g. solution), an acetate-containing formulation (e.g. solution) or a borate-containing formulation (e.g. solution), or a freeze-dried powder.

Active ingredients may further, and/or in the alternative, be combined with appropriate excipients to prepare:
gel formulations (for which suitable gel matrix materials include cellulose derivatives, carbomer and alginates, gummi tragacanthae, gelatin, pectin, carrageenan, gellan gum, starch, Xanthan gum, cationic guar gum, agar, noncellulosic polysaccharides, saccharides such as glucose, glycerin, propanediol, vinyl polymers, acrylic resins, polyvinyl alcohol, carboxyvinyl polymer and, particularly, hyaluronic acid);
lotions (for which suitable matrix materials include cellulose derivatives, glycerin, noncellulosic polysaccharides, polyethylene glycols of different molecular weights and propanediol);
pastes or ointments (for which suitable paste matrix materials include glycerin, vaseline, paraffin, polyethylene glycols of different molecular weights, etc.);
creams or foams (for which suitable excipients (e.g. foaming agents) include hydroxypropyl methyl cellulose, gelatin, polyethylene glycols of different molecular weights, sodium dodecyl sulfate, sodium fatty alcohol polyoxyethylene ether sulfonate, corn gluten powder and acrylamide);
powder aerosols (for which suitable excipients include mannitol, glycine, dextrin, dextrose, sucrose, lactose, sorbitol and polysorbates, e.g. a dry powder inhalant);
liquids, for example water, (aerosol) sprays for oral use or for inhalation (for which suitable excipients include viscosity modifiers, such as hyaluronic acid, sugars, such as glucose and lactose, emulsifiers, buffering agents, alcohols, water, preservatives, sweeteners, flavours, etc.); and/or
injectable solutions or suspensions (which may be aqueous or otherwise and for which suitable excipients include solvents and co-solvents, solubilizing agents, wetting agents, suspending agents, emulsifying agents, thickening agents, chelating agents, antioxidants, reducing agents, antimicrobial preservatives, buffers and/or pH modifiers, bulking agents, protectants and tonicity-modifying agents), particular injectable solutions or suspensions that may be mentioned include dermal fillers (i.e. injectable fillers or soft-tissue fillers).

Moisturizing agents, such as glycerol, glycerin, polyethylene glycol, trehalose, glycerol, petrolatum, paraffin oil, silicone oil, hyaluronic acid and salts (e.g sodium and potassium salts) thereof, octanoic/capyic triglyceride, and the like; and/or antioxidants, such as vitamins and glutathione; and/or pH modifiers, such as acids, bases and pH buffers, may also be included in such formulations, as appropriate. Furthermore, surfactants/emulsifiers, such as hexadecanol (cetyl alcohol), fatty acids (e.g. stearic acid), sodium dodecyl sulfate (sodium lauryl sulfate), sorbitan esters (e.g. sorbitan stearate, sorbitan oleate, etc.), monoacyl glycerides (such as glyceryl monostearate) polyethoxylated alcohols, polyvinyl alcohols, polyol esters, polyoxyethylene alkyl ethers (e.g. polyoxyethylene sorbitan monooleate), polyoxyethylene castor oil derivatives, ethoxylated fatty acid esters, polyoxylglycerides, lauryl dimethyl amine oxide, bile salts (e.g. sodium deoxycholate, sodium cholate), phospholipids, N, N-dimethyldodecylamine-N-oxide, hexadecyltrimethyl-ammonium bromide, poloxamers, lecithin, sterols (e.g. cholesterol), sugar esters, polysorbates, and the like; preservatives, such as phenoxyethanol, ethylhexyl glycerin, and the like; and thickeners, such as acryloyldimethyltaurate/VP copolymer, may be included. In particular stearic acid, glyceryl monostearate, hexadecanol, sorbitan stearate, cetyl alcohol, octanoic/capric glyceride, etc. may be included, particularly in cream formulations.

Formulations of the invention (e.g. aqueous solutions, gels, creams, ointments, lotions, foams, pastes and/or dry powders as described above), may further be combined with an appropriate matrix material to prepare a dressing or a therapeutic patch for application on a biological surface, such as the skin or a mucosal surface. Such formulations may thus be employed to impregnate a matrix material, such as gauze, non-woven cloth or silk paper. The therapeutic patch may alternatively be, for example, a band-aid, a facial mask, an eye mask, a hand mask, a foot mask, etc.

Vaseline may be employed for use in applying such dressings to wounds, but we have also found that ointments based on PEGs (e.g. PEG 400) may be combined with matrix materials to prepare dressings without the need to use vaseline.

LRAs and salts thereof may be readily dissolved in PEG-based solvents to make creams and ointments that have a lower pH (e.g. pH 5.9 to 7.5) rather than the higher pH (pH 9.5) that is observed in mainly aqueous formulations (e.g. water with a very low amount ethanol), and may irritate the skin or mucosal membranes. LRAs/salts be solubilized in this way and then formulated into creams or ointments that are surprisingly stable (i.e. not prone to oxidation), with a pH that is more acceptable/less irritating to the skin/mucosa.

LRAs may be administered for inhalation by way of suspension, a dry powder or a solution. Suitable inhalation devices include pressurized metered-dose inhalers (pMDIs), which may be hand- or breath-actuated and employed with or without a standard spacer device), dry powder inhalers (DPIs), which may be single-dose, multi-dose, and power-assisted, and soft mist inhalers (SMIs) or nebulizers, in which aerosol drug in a fine mist is delivered with slower velocity than a spray delivered using, for example, a pMDI.

In pMDIs, LRAs may be administered as a pressurized suspension of micronized particles distributed in a propellant (e.g. HFA, along with excipients, such as mannitol, lactose, sorbitol, etc.), or as an ethanolic solutions, to deliver one or more metered dose of between about 20 and about 100 μL with each actuation. Actuation may be effected by hand (e.g. pressing) or by inhalation (breath-actuation), involving a flow-triggered system driven by a spring.

In DPIs, LRAs may be administered in the form of micronized drug particles (of a size between about 1 and about 5 μm), either alone or blended with inactive excipient of larger particle size (e.g. mannitol), inside a capsule, which may be pre-loaded or manually loaded into the device. Inhalation from a DPI may de-aggregate the medication particles and disperse them within the airways.

In SMIs, LRAs may be stored as a solution inside a cartridge, which is loaded into the device. A spring may release the dose into a micropump, such that the dose is released when a button is pressed, releasing jet streams of drug solution.

Various nebulizers may also be used to administer LRAs in the form of a fine mist of aerosolized solution. Nebulizers may include breath-enhanced jet nebulizer (in which, with the assistance of a compressor, an air stream moves through jet causing drug solution to be aerosolized); breath-actuated jet nebulizers (in which, after a patient inhales, with the assistance of a compressor, an air stream moves through a tube causing the drug solution to be aerosolized); ultrasonic nebulizers (in which piezoelectric crystals vibrate causing aerosolization by heating causing nebulization); vibrating mesh nebulizers (in which piezoelectric crystals vibrate a mesh plate causing aerosolization to give very fine droplets without a significant change in temperature of the solution during nebulization).

According to a further aspect of the invention there is provided a process for the preparation of a formulation of the invention, as defined herein, which process comprises bringing into association a LRA or salt or solvate thereof, as hereinbefore defined, with one or more pharmaceutically-acceptable excipient, as hereinbefore defined.

LRAs and salts thereof may also be combined in treatment with one or more growth factors selected from platelet-type growth factors (including platelet-derived growth factors, PDGFs); osteosarcoma-derived growth factors (ODGF), epidermal growth factors (EGFs), transforming growth factors (TGFα and TGFβ), fibroblast growth factors (αFGF, βFGF), insulin-like growth factors (IGF-I, IGF-II), nerve growth factors (NGF), interleukin-type growth factors (IL-1, IL-1, IL-3), erythropoietin (EPO), and colony stimulating factor (CSF).

According to a further aspect of the invention there is provided a (e.g. pharmaceutical) formulation of the invention comprising a LRA or a salt or solvate thereof as defined herein, and one or more pharmaceutically-acceptable excipient, such as an adjuvant, diluent or carrier. Preferred formulations are suitable for application locally to e.g. the mucosa (including the oral and/or nasal mucosa, the lung, the anorectal area and/or the colon) or, more preferably, the skin and therefore comprise a topically-acceptable adjuvant, diluent or carrier.

Thus formulations may be suitable for, adapted for, and/or packaged and presented for topical administration (e.g. to the mucosa, including the oral and/or nasal mucosa, the lung, the anorectal area and/or the colon, or, preferably, to the skin), for example the use of such a formulation in the treatment of inflammation, an inflammatory disorder and/or a condition characterised by inflammation (e.g. as a symptom) by way of direct topical administration of that formulation (e.g. to the mucosa, including the oral and/or nasal mucosa, the lung, the anorectal area and/or the colon, or, preferably, to the skin).

In relation to this aspect of the invention, for the avoidance of doubt, topical formulations of the invention may be used in any and all conditions described herein, including treatments of inflammation, in the treatment of any and all inflammatory disorder(s), and/or in the treatment of any and all condition(s) characterised by inflammation, as hereinbefore mentioned, defined or described. Similarly, topical formulations of the invention that may be mentioned include any and all of those mentioned, defined or described herein. Any and all of the relevant disclosures herein are hereby incorporated by reference in conjunction with this aspect of the invention.

Topical (e.g. liquid- or (e.g. aqueous) solution-based) formulations of the invention may be particularly useful in wound recovery, and may alleviate pain (including aching) and, particularly, pruritis/itching that is associated with the wound itself and the wound healing process. Such topical formulations of the invention may be particularly useful in the prevention and/or suppression of the exudation of body fluids from wounds, particularly during the acute inflammation stage, for example during the first 48 hours, after a burn or wound has been inflicted. This prevents the risk of infection, and other physiological reactions. Such topical formulations of the invention may also be particularly useful in the prevention and/or suppression of scarring and melanin pigmentation (vide supra), whether associated with wounds or otherwise.

Administration of formulations of the invention may be continuous or intermittent. The mode of administration may also be determined by the timing and frequency of administration, but is also dependent, in the case of the therapeutic treatment of inflammation, on the severity of the condition.

Depending on the disorder, and the patient, to be treated, as well as the route of administration, LRAs and salts thereof according to the invention may be administered at varying therapeutically effective doses to a patient in need thereof.

Similarly, the amount of active ingredient in a formulation will depend on the severity of the condition, and on the patient, to be treated, but may be determined by the skilled person.

In any event, the medical practitioner, or other skilled person, will be able to determine routinely the actual dosage, which will be most suitable for an individual patient, depending on the severity of the condition and route of administration. The dosages mentioned herein are exemplary of the average case; there can, of course, be individual instances where higher or lower dosage ranges are merited, and such are within the scope of this invention.

Doses may be administered between once and four (e.g. three) times daily.

Appropriate concentrations of LRA/salt thereof in an aqueous solution product may be 0.1 to 10 mg/mL calculated as the free base, and appropriate pH values for such solutions are in the range of about 5.0 (e.g. about 7.0) to about 11.0 (for example about 5.5 (e.g. about 8.0) to about 10.0, such as about pH 9.0 or between about 5.5 to about 7.5), irrespective of how the formulation is prepared.

Appropriate topical doses of LRAs and salts/solvates thereof for administration to the skin are in the range of about 0.01 to about 50 (such as about 20, e.g. about 17.5, including about 10) μg/cm² of treated area, such as about 0.05 (e.g. about 0.1, including about 0.5) to about 17.5, including about 10, such as about 7.5, e.g. about 5) μg/cm² of treated area, in all cases calculated as the free base.

Appropriate doses of LRAs and salts/solvates thereof for nasal administration (e.g. by inhilation) are in the range of about 0.01 μg to about 2000 mg, for example between about 0.1 μg to about 500 mg, or between 1 μg to about 100 mg. Particular doses for nasal administration that may be mentioned include between about 10 μg to about 1 mg, particularly a dose of about 0.1 mg (i.e. about 100 μg). Nasal administration of about 0.1 mg per day of LRAs and salts thereof may be particularly effective in the treatment of conditions associated with inflammation of the nasal passages and mucosae, such as rhinitis (e.g. allergic rhinitis).

Appropriate doses of LRAs and salts/solvates thereof for pulmonary administration (e.g. by inhalation) are in the range of about 0.01 μg to about 2000 mg, for example between about 0.1 μg to about 500 mg, or between 1 μg to about 100 mg. Particular doses for pulmonary administration that may be mentioned include between about 10 μg to about 10 mg, particularly a dose of about 0.6 mg (i.e. 60 μg) to 6 mg (e.g. for use in treating COPD or IPF).

We prefer that pH values of formulations comprising compounds of the invention are in the range of about 1.0 to about 9.0 (for example about 3.0 to about 8.0).

In any event, the dose administered to a mammal, particularly a human, in the context of the present invention should be sufficient to effect a therapeutic response in the mammal over a reasonable timeframe (as described hereinbefore). One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the age, condition, body weight, sex and response of the patient to be treated, and the stage/severity of the disease, as well as genetic differences between patients.

In the uses and methods described herein, LRAs and salts thereof, and formulations comprising them, may also be combined with one or more active ingredients that are useful in the treatment of inflammation and/or inflammatory disorders (other anti-inflammatory agents). Such patients may thus also (and/or already) be receiving therapy based upon administration of one or more of such other active ingredients, by which we mean receiving a prescribed dose of one or more of those active ingredients mentioned herein, prior to, in addition to, and/or following, treatment with a LRA according to the invention.

Such anti-inflammatory agents that may be used in combination with LRAs/salts thereof in the treatment of inflammation include therapeutic agents (e,g, LRAs) (useful in the treatment of inflammation and/or of diseases characterized by inflammation as one of its symptoms. Depending on the condition to be treated, such anti-inflammatory agents may include NSAIDs, corticosteroids, analgesics and certain enzymes, such as trypsin, for example as described hereinafter. Compounds of the invention may also be combined with leukotriene B4 (LTB4).

LRAs and salts thereof as described herein may also be combined for use in the treatment of inflammation with one or more mussel adhesive proteins (MAPs), which includes any adhesive protein that may be derived from mussel species, such as Mytilus edulis (blue mussel), including full length proteins, including all sub-types, that are or may be derived from mussels, such as the collagens pre-COL-P, pre-COL-D and pre-COL-NG, the mussel feet matrix proteins PTMP and DTMP, and, more preferably, mfps or mefps, such as mefp-2, mefp-3, mefp-4, mefp-5, mefp-6 and especially mefp-1, and includes mixtures or combinations of any of these proteins, such as mefps. Although mixtures/combinations of the aforementioned MAP sub-types may be provided as the MAP “component” in accordance with the invention, we prefer that the purity of the principal MAP sub-type (e.g. mefp-1) is at least 25% by weight of the total amount of any such mixture.

Naturally-occurring MAPs may be prepared, for example by mixed adsorption chromatography (see Chinese Patent No. ZL200710179491.0), by carboxymethyl ion exchange chromatography (see Chinese Patent No. ZL200710179492.5), and/or by salting out and dialysis (Chinese Patent No. ZL200910087567.6). Commercial sources of MAPs include USUN Bio Co. (China; sold as MAP Medical Device®), BD Biosciences (USA), Kollodis (South Korea) and Biopolymer (Sweden). MAPs may alternatively be produced using known recombinant DNA methods.

Derivatives of MAPs include isolated (e.g. pharmaceutically-acceptable derivatives), such as lower molecular weight products (for example with a molecular weight in the range of about 500 Da to about 2,000 (e.g. about 1,200, such as about 800) Da, which may allow for easier permeation through biological membranes, such as the skin barrier or a mucosal surface. Such derivatives may also include other compounds that comprise amino acid sequences that are the same as, or are (e.g. minor) variants of, sequences that have been identified in naturally-occurring MAPs, and which may be synthesised by chemical and/or biological processes (e.g. chemical modifications of naturally-occurring MAPs, or direct synthesis). By “(e.g. minor) variants of amino acid sequences identified in naturally-occurring MAPs”, we mean variations in those sequences that do not negatively affect the requisite properties of the relevant naturally-occurring MAP to a measurable degree.

For example, the isolated decapeptide compounds of the sequences:

Ala-Lys-Pro-Ser-Tyr-Hyp-Hyp-Thr-DOPA-Lys (mefp-1 decapeptide, SEQ ID No: 1; see Waite, Int. J. Adhesion and Adhesives, 7, 9 (1987)). MAP Peptide may derived and/or isolated as a low molecular weight derivative of naturally-occurring MAPs, or may be synthesized, for example as described by Yamamoto in J. Chem. Soc., Perkin Trans. 1, 613 (1987). See also Dalsin et al, J. Am. Chem. Soc., 125, 4253 (2003)); and
the analogue Ala-Lys-Pro-Ser-Tyr-Hyp-Hyp-Thr-Tyr-Lys (SEQ ID No: 2; see for example, Kanyalkar et al in Biomaterials, 389 (2002) and Belli et al in Dental Materials, 26, e125 (2010))
are pharmaceutically-acceptable low molecular weight derivatives of MAP that may be combined with LRA or salt thereof according to the invention.

Such derivatives of MAPs may be employed in combination products according to the invention alone, or in combination with one or more other such derivatives, and/or one or more of the aforementioned full length MAPs.

Appropriate concentrations of MAPs and derivatives thereof in an aqueous solution topical formulation product may be about 0.01 (e.g. about 0.1) to about 15.0 (e.g. about 1.5) mg/mL, and appropriate pH values are in the range of about 1.0 to about 7.0 (for example about 3.0 to about 6.5), irrespective of whether the formulation employed is a combined preparation or a kit of parts as hereinbefore described. Suitable commercial sources of such aqueous solutions include USUN Bio Co., Jiangyin, Jiangsu Province, China.

Appropriate topical doses of MAPs and derivatives thereof are in the range of about 0.1 to about 50 μg/cm² of treated area, such as about 1 to about 20 μg/cm² of treated area, including about 2 to about 10 μg/cm² of treated area, such as about 5 μg/cm² of treated area.

Other preferred agents that may be combined with LRAs/salts thereof in accordance with the invention include LTB4 (to treat wounds and burns), and trypsin (to treat inflammation of the mucosa associated with e.g. viral infections).

Formulations of the invention may also be combined with other therapeutic agents which, when administered, are known to give rise to inflammation as a side-effect.

Such combination therapies provide for the administration of at least one LRA or salt thereof as hereinbefore defined in conjunction with one or more such other anti-inflammatory agents, and may thus be presented either as separate formulations, wherein at least one of those formulations comprises LRA/salt, and at least one comprises another anti-inflammatory agent, or may be presented (i.e. formulated) as a combined preparation (i.e. presented as a single formulation including both active ingredients).

Thus, there is further provided:

(1) a formulation (e.g. a pharmaceutical and/or topical formulation) including a LRA or a salt or solvate thereof; another anti-inflammatory agent, or agent known to give rise to inflammation as a side-effect; and a (e.g. pharmaceutically-acceptable) excipient (e.g. adjuvant, diluent or carrier), which formulation is hereinafter referred to as a “combined preparation”; and
(2) a kit of parts comprising components:
(A) formulation (e.g. a pharmaceutical and/or topical formulation) including a LRA or a salt or solvate thereof in admixture with a (e.g. pharmaceutically-acceptable) adjuvant, diluent or carrier; and
(B) a formulation (e.g. a pharmaceutical and/or topical formulation) including another anti-inflammatory agent, or agent known to give rise to inflammation as a side-effect, in admixture with a (e.g. pharmaceutically-acceptable) adjuvant, diluent or carrier,
which components (A) and (B) are each provided in a form that is suitable for administration in conjunction with the other.

According to a further aspect of the invention, there is provided a method of making a kit of parts as defined above, which method comprises bringing component (A), as defined above, into association with a component (B), as defined above, thus rendering the two components suitable for administration in conjunction with each other.

By bringing the two components “into association with” each other, we include that components (A) and (B) of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Thus, there is further provided a kit of parts comprising:

(I) one of components (A) and (B) as defined herein; together with
(II) instructions to use that component in conjunction with the other of the two components.

The kits of parts described herein may comprise more than one formulation including an appropriate quantity/dose of a LRA/salt, and/or more than one formulation including an appropriate quantity/dose of another anti-inflammatory agent, in order to provide for repeat dosing. If more than one formulation (comprising either active compound) is present, such formulations may be the same, or may be different in terms of the dose of either compound, chemical composition(s) and/or physical form(s).

With respect to the kits of parts as described herein, by “administration in conjunction with”, we include that respective formulations comprising a LRA (or salt thereof) and other anti-inflammatory agent are administered, sequentially, separately and/or simultaneously, over the course of treatment of the relevant condition.

Thus, in respect of the combination product according to the invention, the term “administration in conjunction with” includes that the two components of the combination product (LRA and other anti-inflammatory agent) are administered (optionally repeatedly), either together, or sufficiently closely in time, to enable a beneficial effect for the patient, that is greater, over the course of the treatment of the relevant condition, than if either a formulation comprising LRA/salt, or a formulation comprising the other agent, are administered (optionally repeatedly) alone, in the absence of the other component, over the same course of treatment. Determination of whether a combination provides a greater beneficial effect in respect of, and over the course of treatment of, a particular condition will depend upon the condition to be treated or prevented, but may be achieved routinely by the skilled person.

Further, in the context of a kit of parts according to the invention, the term “in conjunction with” includes that one or other of the two formulations may be administered (optionally repeatedly) prior to, after, and/or at the same time as, administration of the other component. When used in this context, the terms “administered simultaneously” and “administered at the same time as” include that individual doses of the relevant LRA/salt and other anti-inflammatory agent are administered within 48 hours (e.g. 24 hours) of each other.

In a further aspect of the invention, there is provided a process for the preparation of a combined preparation as hereinbefore defined, which process comprises bringing into association a LRA or salt thereof, as hereinbefore defined, the other anti-inflammatory agent, or agent known to give rise to inflammation as a side-effect, and at least one (e.g. pharmaceutically-acceptable) excipient.

In relation to the combination product according to the invention, it is preferred that the LRA is not montelukast.

Wherever the word “about” is employed herein, for example in the context of amounts, such as concentrations and/or doses of active ingredients, molecular weights or pHs, it will be appreciated that such variables are approximate and as such may vary by ±10%, for example ±5% and preferably ±2% (e.g. ±1%) from the numbers specified herein. In this respect, the term “about 10%” means e.g. ±10% about the number 10, i.e. between 9% and 11%.

Formulations of the invention have the advantage that they may be used in variety of conditions characterized by inflammation, whether that condition is an organic inflammatory disease per se or is associated with, or is characterized by, inflammation (e.g. a wound, a burn or a viral infection).

The formulations, uses and methods described herein may also have the advantage that, in the treatment of the conditions mentioned hereinbefore, they may be more convenient for the physician and/or patient than, be more efficacious than, be less toxic than, have a broader range of activity than, be more potent than, produce fewer side effects than, or that it/they may have other useful pharmacological properties over, similar formulations or methods (treatments) known in the prior art, whether for use in the treatment of inflammation, inflammatory disorders, or disorders characterized by inflammation as a symptom (including wounds), or otherwise.

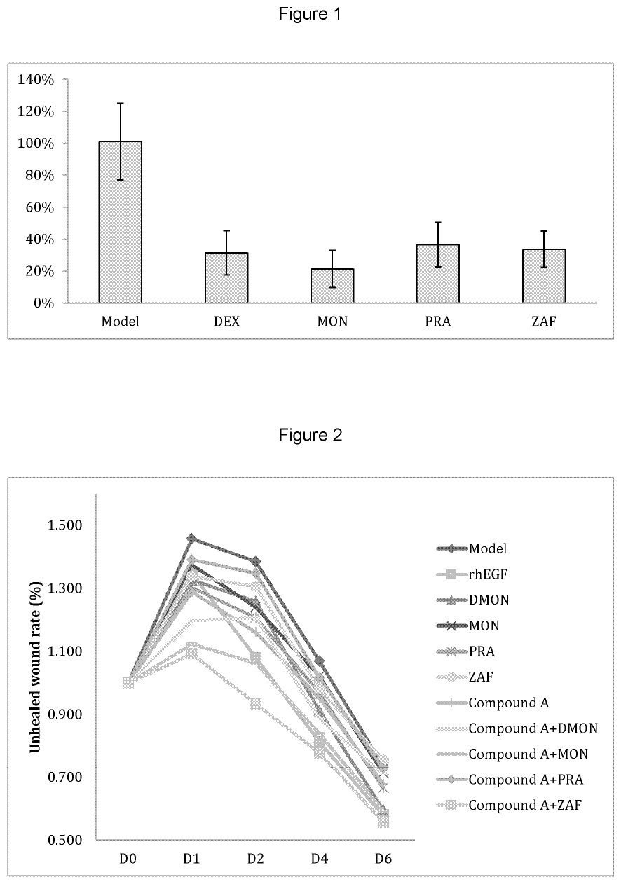

The invention is illustrated by the following examples, in which, for various formulations, including formulations of the invention, FIG. 1 shows the swelling rates in a mouse ear swelling model; and FIG. 2 shows the unhealed wound rate in an acute wound mouse model.

### EXAMPLES

### Example 1

### Synthesis of Ala-Lys-Pro-Ser-Tyr-Hyp-Hyp-Thr-Tyr-Lys

Fmoc-Lys-Boc-Wang resin (0.3 mmol/g; GLS180322-41301, GL Biochem, Shanghai, China) was loaded into a reaction column.

2 litres of methylene chloride (DCM; Shandong Jinling Chemical Industry Inc. Co., Shandong, China) was added to the column and allowed to soak the resin for about half an hour. The DCM was then drawn off and 2 litres of N,N-dimethylformamide (DMF; Shandong Shitaifeng Fertilizer Industry Inc. Co., Shandong, China) was used to wash the column three times.

200 mL of piperidine (Shanghai Li Ming Industry and Trade Co., Ltd., China) was mixed with 1 litre of DMF and was used as deprotection solution. The liquid was drained after 15 minutes and the column was washed with DMF six times.

69 g of Fmoc-Tyr(tBu)-OH (GLS170916-36901, GL Biochem) and 48 g of 2-(1H-benzotriazole-1-yl)-1,1,3,3-tetramethylaminium tetrafluoroborate (TBTU; GL Biochem) were dissolved in 300 mL of DMF and were added to the reaction column. 53 mL of N,N-diisopropylethylamine (DIPEA; Suzhou Highfine Biotech Co. Ltd, Jiangsu, China) was then added. The reaction time was one hour.

A sample was taken, and ninhydrin (Shanghai Shanpu Chemical Co. Ltd, China) was used to detect when the reaction was complete. At this point, the liquid was drained and the residue was washed three times with DMF.

The above coupling steps were repeated to couple the remaining amino acids in the same amounts: Fmoc-Thr(tBu)-OH, Fmoc-4-Hyp(tBu)-OH, Fmoc-4-Hyp(tBu)-OH, Fmoc-Tyr(tBu)-OH, Fmoc-Ser(tBu)-OH, Fmoc-Pro-OH, Fmoc-Lys(Boc)-OH and Fmoc-Ala-OH.

At the end of the reaction sequence, 20% piperidine in DMF was added as a deprotection solution in the manner described above. The liquid was then drained after 15 minutes and the column washed with DMF, DCM and methanol, 3 times each, respectively.

The liquid was drained to obtain the resin-bound polypeptide.

An appropriate amount of lysate, which was comprised of 95% trifluoroacetic acid (TFA), 2.5% water and 2.5% triisopropylsilane (Tis), was added to immerse the resin-bounded polypeptide. The mixture was put on a shaker and incubated for 2 hours at 30 to 35° C. The resin was then removed by filtration.

Anhydrous ice ether was added into the filtrate, which was centrifuged and the supernatant discarded. The resultant was washed with anhydrous ice ether three times. The isolated peptide was dried by desiccation to obtain 128 g of crude polypeptide, which was further purified to provide 70.7 g of purified peptide, which was used in the experiments described below.

LCMS (column: GS-120-5-C18-BIO; detection: UV at 220 nm; solvent A: 0.1% TFA in MeCN, solvent A: 0.1% TFA in water; flow rate 1.0 mL/min.; volume: 10 μL): m/z 592.65 [M+2H]2+ at 14.351 min. (97.89%).

The compound of Example 1 is referred to hereinafter as “Compound A”.

### Example 2

### Mouse Ear Swelling Model

35 healthy male BALB/c mice of 6-8 weeks of age and average body weight of 18-25 g supplied by Changzhou Cvens Experimental Animal Co. Ltd. were housed and cared for about for 1 week prior to the experiment. The housing temperature was 25-27° C. with 74% humidity, with alternating 12 hour periods of light and darkness, and free access to food and water. The mice were randomly divided into 4 groups as described in Table 1 below, with 5 mice in each group.

The left ear of each mouse was used as autologous control. The right ear of each mouse was treated by various different treatments, as summarised in Table 1 below. 20 μL of xylene (Shanghai Aladdin Bio-Chem Technology Co., Ltd., Shanghai, China) was applied to the right ear of each mouse, both inside and outside. The ear started to swell in about 4 minutes. Then, different amounts of each study treatments or vehicles (as described in Table 1 below) were applied to the right ears in each group. The mice were put back into their cages.

A base cream was made according to the following procedure.

A first mixture was made, consisting of the following components: sorbitan stearate (0.6 g), polysorbate-80 (1 g), hexadecanol (2 g), octanoic acid/decanoic acid glyceride (5 g), liquid paraffin (4 g), monostearate glyceride (2 g) and vaseline (5 g) (all Sinopharm Chemical Reagent Co. Ltd.). The components were mixed together and heating to 85° C. with stirring until the mixture melted completely.

A second mixture was also made, consisting of the following components: methyl cellulose (0.5 g), glycerin (4 g), trehalose (0.5 g), polyethylene glycol 200 (4 g), phenoxyethanol (0.3 g) and ethylhexyl glycerol (0.1 g) (all Sinopharm Chemical Reagent Co. Ltd.) and purified water (69.45 g). The components were mixed together and heating to 85° C. with stirring to form a homogeneous colloidal suspension.

The sorbitan stearat-containing mixture and the methyl cellulose-containing mixture were combined. Silicone oil (0.5 g) was added to the resultant mixture, which was then emulsified by stirring quickly for five minutes using emulfication equipment. The resultant emulsion was cooled to 55° C., to give the base cream. A cream based on montelukast (MON) was made using the following procedure. Montelukast sodium (14 mg; Arromax Pharmatech Co., Ltd, Suzhou, China), was dissolved in DMSO (0.6 mL Shanghai Aladdin Bio-Chem Technology Co., Ltd, Shanghai, China). The montelukast solution was added to 2.2 g of the base cream and the mixture stirred until uniform.

Zafirlukast cream (ZAF) was made using the same procedure, except that montelukast was replaced by zafirlukast (50 mg; Shanghai Aladdin Bio-Chem Technology Co., Ltd, Shanghai, China) and 9.4 g of the base cream was used.

Pranlukast cream (PRA) was made using the same procedure as for the zafirlukast cream, except that zafirlukast was replaced by pranlukast hemihydrate (50 mg; Anhui Heryi pharmaceutical Co., Ltd, Anhui, China)

Dexamethasone cream (DEX), was prepared by adding dexamethasone acetate (5 mg; Fuyuan Pharmaceutical Co. Ltd., Anhui, China) with 10 g of the base cream and the mixture stirred until uniform. The dexamethasone cream was used as the positive control.

TABLE 1

| | | Drug | |
|---|---|---|---|
| | Drug | administration | Total amount of |
| Group | concentration | on right ear | drugs (μg/mouse) |
| Model | / | xylene | / |
| DEX | 5 mg/10 g | xylene + DEX | 400 |
| | | cream | |
| MON | 5 mg/g | xylene + MON | 500 |
| | | cream | |
| ZAF | 5 mg/g | xylene + ZAF | 500 |
| | | cream | |
| PRA | 5 mg/g | xylene + PRA | 500 |
| | | cream | |

The mice were sacrificed by cervical dislocation after 40 minutes. The left and right ears were cut off. A skin pouch (Electron Microscopy Sciences, Hatfield, USA) with diameter of 8 mm was used to take a piece of ear from the same site of both ears. The weights were recorded the weights and the swelling rates were calculated as follows:

Swelling rate=(right ear weight−left ear weight)/left ear weight×100%

and the results are shown in FIG. 1.

The results show that the montelukast, zafirlukast and pranlukast creams significantly reduce the xylene induced swelling.

### Example 3

### Acute Wound Model

6-8 weeks old male C57BL/6 mice were supplied by Changzhou Cvens Experimental Animal Co. Ltd. Prior to any experiments being conducted, mice were housed under standardized conditions (at a constant temperature or 22±2° C., with alternating 12-hour periods of light and darkness) and were fed on a standard mouse diet with water, for about a week.

General anesthesia was induced using intraperitoneal 3% chloral hydrate (Sinopharm Chemical Reagent Co., Ltd.; 1 mL/10 g of body weight). The hair on the back was shaved by a baby hair shaver and depilated with cream. The skin area was wiped and sterilized with 75% alcohol twice.

EMS skin biopsy punch (Electron Microscopy Sciences, P.O. Box 550, 1560 Industry Road, Hatfield, Pa. 19440) with a 12 mm diameter was used to make two adjacent round wounds on the midline of the back. The two circles were tangential to each other and the skin between the circles was cut along the upper and lower tangents. Scissors were used to trim the wound. The wound was an oval shape. The full thickness of skin was removed and the depth reached the fascia. The wounds were left open without suture.

Compound A (see Example 1 above) was obtained as a powder from GL Biochem. Hydrogels of Compound A (A) were prepared consisting of the following components: active ingredients in the amounts of described in Table 2 below, methyl cellulose (2.5%), propanediol (11%), glycerol (11%) and 1% acetic acid (pH regulator; 0 to 0.5 g). All excipients were obtained from Sinopharm Chemical Reagent Co. Ltd. The gels were made up with water for injection. The amount of water for injection varied and could be calculated by (100%-2.5%-11%-11%-W_{acetic acid} %-W_{compound A} %).

A base cream was made according to the following procedure described in Example 2.

A cream based on montelukast was made (MON) using the procedure described in Example 2 above except that 9.4 g of the base cream was employed.

A cream based on montelukast styrene (obtained from National Institute for Food and Drug Control, China) (DMON) was made using the same procedure, except that montelukast was replaced by montelukast impurity B (14 mg; pharmaceutical standard material, National Institutes for Food and Drug Control (NIFDC), China) and 2.2 g of the base cream was used.

Zafirlukast cream (ZAF) was made using the same procedure, except that montelukast was replaced by zafirlukast (50 mg; Shanghai Aladdin Bio-Chem Technology Co., Ltd, Shanghai, China) and 9.4 g of the base cream was used.

Pranlukast cream (PRA) was made using the same procedure as for the zafirlukast cream, except that zafirlukast was replaced by pranlukast (50 mg; Anhui Heryi pharmaceutical Co., Ltd, Anhui, China)

Different drugs were administrated topically at 50 μL/wound, once daily from Day 0 to Day 7. The hydrogel and cream were put into a 1 mL syringe to measure the dose based on calibration of weight and volume. The model group was given same amount of normal saline. There were 11 groups including 88 mice in this experiment, as shown in Table 2 below.

Recombinant Human Epidermal Growth Factor (rhEGF, Shanghai Haohai Biological Technology Co. Ltd, Shanghai, China) was purchased and prepared according to the manufacturer's instructions. Lyophilized rhEGF powder (100000 IU/vial) was dissolved in 20 mL of normal saline to make a solution with a 5000 IU/mL concentration. The working dose of rhEGF for this experiment was 1285 IU/wound.

The hydrogel of Compound A and the cream of the four other drugs were mixed together prior to administration and then applied to the wound of the animals in the combination therapy groups.

TABLE 2

| Group | Meaning | Dose (/wound/day) | |
|---|---|---|---|
| Model | C57 mice with wounds/ | / | |
| | normal saline | | |
| rhEGF | C57 mice with wounds/ | 1285 | IU |
| | rhEGF | | |
| A | C57 mice with wounds/ | 12.25 | μg |
| | Compound A gel | | |
| MON | C57 mice with wounds/ | 200 | μg |
| | montelukast sodium cream | | |
| DMON | C57 mice with wounds/ | 200 | μg |
| | montelukast styrene cream | | |
| ZAF | C57 mice with wounds/ | 200 | μg |
| | zafirlukast cream | | |
| PRA | C57 mice with wounds/ | 200 | μg |
| | pranlukast cream | | |

| | | |
|---|---|---|
| A + MON | C57 mice with wounds/ | 6.13 μg Compound A + |
| | Compound A gel and | 100 μg MON |
| | montelukast cream | |
| A + DMON | C57 mice with wounds/ | 6.13 μg Compound A + |
| | Compound A gel and | 100 μg DMON |
| | montelukast styrene cream | |
| A + ZAF | C57 mice with wounds/ | 6.13 μg Compound A + |
| | Compound A gel and | 100 μg ZAF |
| | zafirlukast cream | |
| A + PRA | C57 mice with wounds/ | 6.13 μg Compound A + |
| | Compound A gel and | 100 μg PRA |
| | pranlukast cream | |

Photographs were taken for each wound every other day from Day 0 and scanned into a computer. The wound areas calculated using ImageJ image analysis software (National Institute of Health, USA).

The unhealed wound area was expressed as a percentage of the original wound area:

Aₜ/A₀×100%,

where A₀ and Aₜ refer to the initial area at Day 0 and the wound area at the date of measurement (time t), respectively.

The unhealed wound rates are shown in FIG. 2. The results showed that all drug formulations promote wound healing effects. The combination groups had the best performance. The combination of Compound A hydrogel plus zafirlukast cream promoted the best recovery rate, 47% faster than that of the model group.

### Example 4

### Idiopathic Pulmonary Fibrosis (IPF) Model

50 Adult SD rats (Zhejiang Experimental Animal Center (Animal Production License Number: SCXK (Zhejiang, Chi-00, half male and half female were kept at 21-26° C. with 40-70% relative humidity, with free access to food and water.

After 7 days of adaptive feeding, the rats were randomly divided into 5 groups: sham operation group, idiopathic pulmonary fibrosis model group, Monte gavage administrating group, Monte inhalation administrating group and pirfenidone control group.

The rats were anaesthetized and placed on an operating table in the supine position, exposing the trachea. 150 μL of Bleomycin (Bleomycin Hydrochloride for Injection, Zhejiang Hisun Pharmaceutical Co. Ltd, China) in saline solution was injected into the trachea through the gap between the tracheal cartilage at 5 mg/kg. (The sham-operation group were given an equal volume of normal saline.)

The rats were lifted vertically after administration and were rotated to allow the drug to evenly disperse. Once the rats recovered, after approximately 7 days, they were administrated different drugs according to Table 1 below.

TABLE 1

| Group | Treatment | Dose | | Volume |
|---|---|---|---|---|
| Sham-operation | saline | 50 | μL | 0.15 mL |
| IPF model group | saline | 50 | μL | 0.15 mL |
| Positive control | pirfenidone | 120 | mg/kg | 1 mL/200 g |
| Monte oral | montelukast | 10 | mg/kg | 1 mL/200 g |
| | sodium | | | |
| Monte inhalation | montelukast | 65 | μg/rat | 0.15 mL |
| | sodium | | | |

In the Montelukast inhalation group, montelukast sodium was atomized using a YLS-8B Multifunctional Cough and Asthma Inducer (Jinan Yiyan Technology Development Co., Ltd, China), and administered via inhalation (0.15 mL/min) for 1 minute, once daily for 28 days, In the other groups, montelukast sodium and pirfenidone (Etuary®, Beijing Continent Pharmaceutical Co., Ltd., Beijing, China) were administered by intragastric administration for 28 days.

The following observation indicators were investigated.
1) General observations of the rats' activity, sensitivity to external stimuli, fur luster, hair colour, mouth, lip, nose, weight, diet, breathing and mortality (daily).
2) Determination of the rats' lung organ coefficient and lung dry-wet weight ratio (i.e. the ratio of the animal's lung weight organ to the animal's body weight, i.e. the ratio of viscera to body weight).
3) During the formation of pulmonary fibrosis, the expression of growth factor (TGF-β), tumour necrosis factor-α (TNF-α) and other cytokines which are involved in the onset of fibrosis. Standard ELISA methods were used to detect the contents of TGF-β, TNF-α, IL-1β, malondialdehyde (MDA) and the activity of superoxide dismutase (SOD) in lung tissue.
4) Detection of the content of collagen and fibrin metabolite (hydroxyproline, HYP) in the lungs, as specific indicators for evaluating the degree of pulmonary fibrosis.
5) Detection of histopathological changes in lung tissue, which is the most important and objective indicator for evaluating pulmonary fibrosis.

The results of effects of montelukast on lung coefficient and HYP, MDA, SOD contents in bleomycin-induced pulmonary fibrosis rats are shown in Table 2 below.

TABLE 2

| | lung | HYP | MDA | SOD |
|---|---|---|---|---|
| | coefficient | (μg/g wet | (U/mg | (U/mg |
| | (%) | weight) | protein) | protein) |
| Sham- | 4.85 ± 0.26 | 527.12 ± 34.82 | 0.12 ± 0.02 | 51.22 ± 1.07 |
| operation | | | | |
| Model | 6.49 ± 0.69 | 1245.06 ± 65.20 | 0.66 ± 0.08 | 41.89 ± 2.82 |
| group | | | | |
| Monte | 5.51 ± 0.36 | 569.99 ± 18.81 | 0.25 ± 0.13 | 49.13 ± 3.56 |
| oral | | | | |
| Monte | 5.56 ± 0.53 | 785.59 ± 63.37 | 0.44 ± 0.09 | 44.14 ± 1.77 |
| inhalation | | | | |
| Postitive | 5.54 ± 0.51 | 755.49 ± 66.64 | 0.24 ± 0.05 | 48.59 ± 2.36 |
| Control | | | | |

The above results indicated that montelukast, when administered by both the oral and the inhalation route, eliminates the pulmonary edema response in bleomycin-induced pulmonary fibrosis in rats, manifested by reduced dry-wet weight ratio of lung tissue and pulmonary coefficient. The synthesis of HYP, the main component of collagen synthesis, was decreased in both montelukast groups, indicating anti-fibrotic efficacy.

The effect of montelukast sodium on TNF-α,IL-10,α-SMA,TGF-β1 contents in tissues are shown in Table 3 below.

TABLE 3

| | TNF-α | IL-10 | α-SMA | TGF-β1 |
|---|---|---|---|---|
| | (pg/mg | (pg/mg | (pg/mg | (ng/mg |
| | protein) | protein) | protein) | protein) |
| Sham- | 181.85 ± 27.34 | 42.73 ± 1.60 | 9.37 ± 0.72 | 0.4 ± 0.06 |
| operation | | | | |
| Model | 301.69 ± 28.97 | 59.50 ± 5.92 | 13.71 ± 1.41 | 0.55 ± 0.05 |
| group | | | | |
| Monte | 220.74 ± 26.79 | 45.45 ± 3.90 | 11.59 ± 2.90 | 0.45 ± 0.03 |
| oral | | | | |
| Monte | 182.24 ± 25.27 | 38.49 ± 2.08 | 9.08 ± 0.89 | 0.43 ± 0.02 |
| inhalation | | | | |
| Positive | 208.41 ± 17.73 | 43.52 ± 4.30 | 10.98 ± 0.93 | 0.41 ± 0.03 |
| Control | | | | |

The production of inflammatory cytokines such as TNF-α, IL-10, and factors related to fibrosis like α-SMA and IL-1 β were decreased in both montelukast groups.

The results of the histopathological examinations showed that the degree of histological injury was significantly reduced, as manifested by the decreases in the scores of tissue injury, the alleviation of bronchial pneumonia and the alleviation of fibrotic tissue proliferation around the bronchi.

### Example 5

### Montelukast Gel for Colitis and Constipation

The patient is a young woman in her 30s, who has been suffering from severe constipation (caused by slow peristalsis of the intestines) for more than 10 years and is diagnosed with colitis with internal and external hemorrhoids. Although, there is no blood in the stool, no stomach pain nor any other discomfort, long-term constipation makes the patient very uncomfortable. The patient frequently visits the community medical center, and is consuming lactulose, wheat bran and refrigerated Lactobacillus. The doctor prescribed lidocaine, gentamicin, and metronidazole enema in the morning and traditional Chinese medicine Huangbai liquid in the afternoon for one week. At the second week, the doctor prescribed western medicine and traditional Chinese medicine enema alternately, rotating every day. Although, enema did improve the bowl movement, it did not cure the constipation.

After using montelukast gel (5 mg/g, 2 g gel in one package; see below) daily for about one month, the patient stopped using the enema and consuming Lactobacillus and was able to defecate daily. Now, the patient only needs to use the gel once or twice when feeling uncomfortable.

The montelukast gel was prepared according to the following procedure.

0.5 g of montelukast sodium (Tianyu Pharmaceutical Co. Zhejiang, China) was dissolved in 42.2 g of distilled water and then 20.0 g of hydroxypropyl-beta-cyclodextrin (HP-β-CD, Shandong Binzhou Zhiyuan Biotechnology Co., Ltd.) was added slowly and the mixture was stirred until it was uniform. 24 g of hydroxypropyl methylcellulose (HPMC, Rohm Haas Electronic Materials (Shanghai) Co., Ltd.) 5% aqueous solution was added and the mixture was mixed well.

3.4 g of potassium dihydrogen phosphate (China Pharmaceutical Group Chemical Reagents Co., Ltd) and 0.8 g of sodium hydroxide (China Pharmaceutical Group Chemical Reagents Co., Ltd) were dissolved in 500 g of distilled water to make a buffer solution with pH of 7.30-7.50.

0.3 g EDTA-2Na (China Pharmaceutical Group Chemical Reagents Co., Ltd) was dissolved in 36.67 g of the above-mentioned buffer solution, and then 3.0 g of sodium thiosulfate and 30 mg of benzalkonium chloride (China Pharmaceutical Group Chemical Reagents Co., Ltd) were dissolved into the mixture.

13.3 g of the above-mentioned mixture was added into the Montelukast solution under constant stirring. This formulation was put aside for resting until all of the bubbles had disappeared.

1. A formulation suitable for, adapted for, and/or packaged and presented for, topical administration, comprising a leukotriene receptor antagonist or a salt or solvate thereof, in admixture with a topical adjuvant, diluent or carrier.

2. The formulation as claimed in claim 1 wherein the leukotriene receptor antagonist is selected from the group cinalukast, pobilukast, pranlukast and zafirlukast.

3. The formulation as claimed in claim 1 wherein the leukotriene receptor antagonist is a compound of formula I: 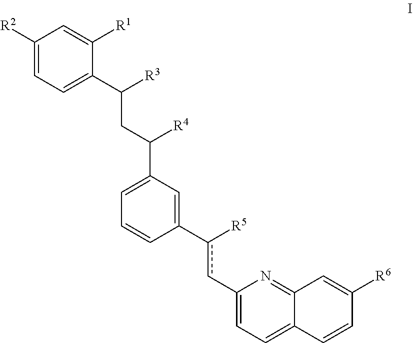
wherein:
R¹ is selected from the group consisting of —C(CH₃)₂OR⁷, —C(═O)CH₃, —C(CH₃)═CH₂, —C(CH₃)₂H, —C(CH₃)(OH)CO₂H, —C(CH₃)(OH)CH₂OH and —C(═O)NH₂;
R² and R³ are independently H or —OH;
R⁴ is H, —OH, —OS(O)₂CH₃, a structural fragment of formula II: 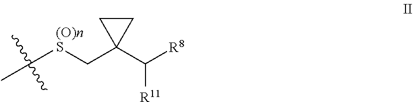
or a structural fragment of formula III: 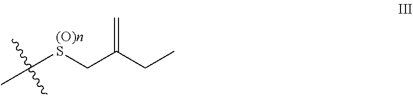
wherein the squiggly lines in the fragments of formula II and III represent the points of attachment of the respective fragments to the compound of formula I, and n is 0, 1 or 2;
the dotted line in the compound of formula I represents an optional double bond and, when a double bond is present, R⁵ represents H and, when a double bond is not present, R⁵ represents H or a structural fragment of formula II as defined above;
R⁶ is H or Cl;
R⁸ is selected from the group consisting of —C(O)R⁹, —CN and a structural fragment of formula IV: 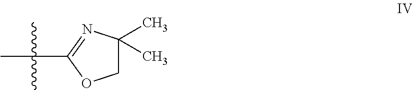 [SMILES: *C1=NC(C)(C)CO1]
wherein the squiggly line in the fragment of formula IV represent the point of attachment to the structural fragment of formula II,
R⁹ is —NH₂ or —OR¹⁰;
R¹¹ is H or OH; and
R⁷ and R¹⁰ are independently H, —CH₃ or a glucuronide residue,
or a regioisomer, a geomeric isomer, or a stereoisomer, thereof.

4. The formulation as claimed in claim 3, wherein, in the compound of formula I, R⁴ is a structural fragment of formula II and is in the following configuration: 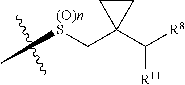
wherein n, R⁸ and R¹¹ are as defined in claim 3.

5. The formulation as claimed in claim 3, wherein, in the compound of formula I, R⁴ represents a structural fragment of formula II or III and n is 0.

6. The formulation as claimed in claim 3, wherein, in the compound of formula I, R⁴ represents a structural fragment of formula II and R¹¹ is H.

7. The formulation as claimed in claim 3, wherein, in the compound of formula I, R⁴ represents a structural fragment of formula II and R⁸ represents —C(O)R⁹.

8. The formulation as claimed in claim 7, wherein R⁹ is —OH.

9. The formulation as claimed in claim 3, wherein, in the compound of formula I, le is selected from the group consisting of —C(CH₃)₂OR⁷, —C(═O)CH₃, —C(CH₃)═CH₂ and —C(CH₃)₂H.

10. The formulation as claimed in claim 9, wherein R¹ is selected from the group consisting of —C(CH₃)₂OH, —C(═O)CH₃, —C(CH₃)═CH₂ and —C(CH₃)₂H.

11. The formulation as claimed in claim 3, wherein, in the compound of formula I, R² is H.

12. The formulation as claimed in claim 3, wherein, in the compound of formula I, R³ is H.

13. The formulation as claimed in claim 3, wherein, in the compound of formula I, the dotted line represents a double bond and the quinoline ring is located trans across the double bond to the central 1,3-disubstituted phenyl ring.

14. The formulation as claimed in claim 3, wherein, in the compound of formula I, R⁶ is Cl.

15. The formulation as claimed in claim 3, wherein, in the compound of formula I, R¹ is —C(CH₃)OR⁷.

16. The formulation as claimed in claim 15, wherein R⁷ is H.

17. The formulation as claimed in claim 3, wherein, in the compound of formula I, R¹ is —C(CH₃)═CH₂ or —C(CH₃)H.

18. The formulation as claimed in claim 3, wherein the compound of formula I is montelukast styrene.

19. The formulation as claimed in claim 3, wherein the compound of formula I is a hydrogenated montelukast styrene.

20. The formulation as claimed in claim 3, which is a pharmaceutical formulation and the topical adjuvant, diluent or carrier is a pharmaceutically-acceptable topical adjuvant, diluent or carrier.

21-22. (canceled)

23. The formulation as claimed in claim 1, wherein the formulation is in the form of a cream or an ointment.

24. The formulation as claimed in claim 23, which comprises a polyethylene glycol.

25. The formulation as claimed in claim 24, wherein the polyethylene glycol is polyethylene glycol 400.

26. A combination product comprising:
(a) at least one mussel adhesive protein or a derivative thereof; and
(b) a leukotriene receptor antagonist, or a pharmaceutically-acceptable salt or solvate thereof.

27. The combination product as claimed in claim 26 which comprises a pharmaceutical formulation including at least one mussel adhesive protein or a derivative thereof; a leukotriene receptor antagonist, or a pharmaceutically-acceptable salt or solvate thereof; and a pharmaceutically-acceptable adjuvant, diluent or carrier.

28. The combination product as claimed in claim 26, which comprises a kit of parts comprising components:
(A) a pharmaceutical formulation including at least one mussel adhesive protein or a derivative thereof, in admixture with a pharmaceutically-acceptable adjuvant, diluent or carrier; and
(B) a pharmaceutical formulation including a leukotriene receptor antagonist, or a pharmaceutically-acceptable salt or solvate thereof, in admixture with a pharmaceutically-acceptable adjuvant, diluent or carrier,
which components (A) and (B) are each provided in a form that is suitable for administration in conjunction with the other.

29. A kit of parts comprising:
(I) one of components (A) and (B) as defined in claim 28; together with
(II) instructions to use that component in conjunction with the other of the two components.

30. The kit of parts as claimed in claim 29, wherein components (A) and (B) are suitable for sequential, separate and/or simultaneous use in the treatment of:
(i) an inflammatory disorder; or
(ii) viral infections or diseases.

31. The combination product as defined in claim 26 wherein the at least one mussel adhesive protein comprises mefp-1.

32. The combination product as defined in claim 26 comprising a derivative of a mussel adhesive protein, which is a peptide of the sequence Ala-Lys-Pro-Ser-Tyr-Hyp-Hyp-Thr-DOPA-Lys or a salt thereof, or Ala-Lys-Pro-Ser-Tyr-Hyp-Hyp-Thr-Tyr-Lys or a salt thereof.

33. The combination product as defined in claim 26, wherein one or more of the formulation(s) are in the form of a cream or an ointment.

34. The formulation as defined in claim 1, wherein the leukotriene receptor antagonist is not montelukast.

35-36. (canceled)

37. A method of treatment of:
(i) inflammation, of an inflammatory disorder, and/or of a disorder or condition characterized by inflammation; or
(ii) viral infections or viral diseases,
which method comprises the administration of a formulation as defined in claim 1 to a patient in need of such treatment.

38. The method as claimed in claim 37, wherein the inflammatory disorder is selected from psoriasis, acne, eczema, dermatitis, rhinitis, pharyngitis, and chronic obstructive pulmonary disease.

39. The method as claimed in claim 38, wherein the dermatitis is atopic dermatitis or steroid-dependent dermatitis.

40. The method as claimed in claim 37, wherein the condition or disorder characterized by inflammation is a wound or a burn.

41. The method as claimed in claim 40, wherein the wound is an abrasion, a scratch, an incision, a laceration, a skin puncture, an avulsion, a bruise, a scar or a blister, or itching associated with any of the foregoing.

42. The method as claimed in claim 37, wherein the condition or disorder characterized by inflammation is colitis, inflammatory bowel disease, Crohn's disease, ulcerative colitis, gastrohelcosis, gastritis, gastric ulcer, gastric cancer, constipation, gastritis, inflammation associated with cancers and/or infections that affect the gastrointestinal tract, gastroesophageal reflux disease, or hemorrhoids.

43. The method as claimed in claim 37, wherein condition is treated by way of direct topical administration to relevant site of inflammation.

44. The method as claimed in claim 43, wherein the administration is to the skin.

45. method as claimed in claim 43, wherein the administration is to a mucosal surface.

46-47. (canceled)

48. A method of treatment of idiopathic pulmonary fibrosis, which method comprises the administration of a formulation as defined in claim 1, to a patient in need of such treatment.

49. The method as claimed in claim 48, wherein the administration is topical.

50. The method as claimed in claim 49, wherein the administration is direct topical administration to the lung.

51. A process for the preparation of a pharmaceutical formulation as defined in claim 1, which process comprises bringing into association the leukotriene receptor antagonist with the one or more adjuvant, diluent or carrier.

52. A process for the preparation of a kit of parts as defined in claim 29, which process comprises bringing into association component (A) of the kit of parts with component (B) of the kit of parts.

53. The method as claimed in claim 37, wherein the viral infections or viral diseases are caused by viruses selected from the group of adenoviridae, papillomaviridae, polyomaviridae, herpesviridae, poxviridae, hepadnaviridae, parvoviridae, astroviridae, caliciviridae, picornaviridae, coronoviridae, flaviviridae, retroviridae, togaviridae, arenaviridae, bunyaviridae, filoviridae, orthomyxoviridae, paramyxoviridae, rhabdoviridae, hepeviridae, reoviridae, hepatitis D, or combinations thereof.
